SEQUENCE LISTING (14 sequences)

SEQ ID NO: 1 - DNA, 16 bp, synthetic construct
  source 1..16: mol_type = other DNA
agagctgacttgatgc

SEQ ID NO: 2 - DNA, 15 bp, synthetic construct
  source 1..15: mol_type = other DNA
agagctgacttgatg

SEQ ID NO: 3 - skipped

SEQ ID NO: 4 - DNA, 24 bp, synthetic construct
  source 1..24: mol_type = other DNA
agagtggttgtggtagcatcaagt

SEQ ID NO: 5 - DNA, 66 bp, synthetic construct
  source 1..66: mol_type = other DNA
cagctcaaaaaaaaaaaaaaaaaaaaaaaaaaaaaaaaaaaaaaaaaaaaaaaaaaaaaaaaaaaa

SEQ ID NO: 6 - DNA, 46 bp, synthetic construct
  source 1..46: mol_type = other DNA
caacaatcaggacgataccgatgaagtttttttttttttttttttt

SEQ ID NO: 7 - DNA, 46 bp, synthetic construct
  source 1..46: mol_type = other DNA
gtcctgattgttgcttcatcggtatcaaaaaaaaaaaaaaaaaaaa

SEQ ID NO: 8 - DNA, 14 bp, synthetic construct
  source 1..14: mol_type = other DNA
tggtagcatcaagt

SEQ ID NO: 9 - DNA, 19 bp, synthetic construct
  source 1..19: mol_type = other DNA
ggttgtggtagcatcaagt

SEQ ID NO: 10 - DNA, 29 bp, synthetic construct
  source 1..29: mol_type = other DNA
tggtaagagtggttgtggtagcatcaagt

SEQ ID NO: 11 - DNA, 34 bp, synthetic construct
  source 1..34: mol_type = other DNA
agagttggtaagagtggttgtggtagcatcaagt

SEQ ID NO: 12 - DNA, 39 bp, synthetic construct
  source 1..39: mol_type = other DNA
ttgtgagagttggtaagagtggttgtggtagcatcaagt

SEQ ID NO: 13 - DNA, 59 bp, synthetic construct
  source 1..59: mol_type = other DNA
tggtaagagtggttgtggtattgtgagagttggtaagagtggttgtggtagcatcaagt

SEQ ID NO: 14 - DNA, 99 bp, synthetic construct
  source 1..99: mol_type = other DNA
ggttgtggtattgtgagagttggtaagagtggttgtggtatggtaagagtggttgtggtattgtgagagttggtaagagtggttgtggtagcatcaagt

(19) United States
(12) Patent Application Publication
(10) Pub. No.: US 2025/0188520 A1
(43) Pub. Date: Jun. 12, 2025

(54) METHOD FOR DETECTING OLIGONUCLEOTIDE WITH SUPPRESSED CROSS-REACTIVITY

(71) Applicant: SEKISUI MEDICAL CO., LTD., Tokyo (JP)

(72) Inventors: Masako OSAWA, Tokyo (JP); Takurou AKIYA, Tokyo (JP)

(73) Assignee: SEKISUI MEDICAL CO., LTD., Tokyo (JP)

(21) Appl. No.: 18/844,787

(22) PCT Filed: Mar. 6, 2023
(86) PCT No.: PCT/JP2023/008244

§ 371 Date: Sep. 6, 2024

(30) Foreign Application Priority Data
Mar. 8, 2022 (JP) 2022-035004
Mar. 8, 2022 (JP) 2022-035006

(51) Int. Cl. C12Q 1/6834; C12Q 1/6876

(52) U.S. Cl. CPC C12Q 1/6834; C12Q 1/6876

(57) ABSTRACT

A method for measuring an oligonucleotide which is simpler and more sensitive and has excellent specificity and quantitative capability compared to the conventional measurement method is provided. Moreover, a method for measuring an oligonucleotide having excellent specificity which can distinguish the intact target oligonucleotide (unchanged form) and a metabolite thereof and detect the unchanged form only is provided. In a hybridization method using a capture probe and an assist probe, by inserting a spacer between a solid phase and a nucleic acid probe contained in the capture probe, it becomes possible not only to detect the target oligonucleotide in a sample but also to distinguish from a metabolite of a nucleic acid drug.

### TECHNICAL FIELD

The present invention relates to a method for detecting or quantifying an oligonucleotide which is highly sensitive and has excellent specificity and quantitative capability.

### BACKGROUND ART

Nucleic acid drugs cause sequence-specific gene silencing and thus recently attract considerable attention as novel therapeutic agents for various diseases which have been difficult so far to treat including genetic diseases and refractory diseases (Non-Patent Document 1, Ando 2012).

In pharmacokinetics/pharmacodynamics (PK/PD) screening tests in the exploratory stage of drug development, in safety tests, pharmacological tests and pharmacokinetics tests in the non-clinical stage and in the clinical stage, drug concentrations in biological samples of animals or humans to which a drug has been administered are measured. Moreover, when a new drug is to be approved, it is required to acquire data on the drug concentrations in biological samples and submit documents regarding the safety and the pharmacokinetics in accordance with the guidelines provided by the ministerial ordinance of the Ministry of Health, Labour and Welfare.

Ando et al. have reported that, by labeling a nucleic acid drug by a positron-emitting nuclide, the in vivo behavior is three dimensionally analyzed noninvasively in real time (Non-Patent Document 1, Ando 2012). Moreover, Healey et al. have reported that, using the stem-loop RT-PCR method (Non-Patent Document 3, Chen 2005), a lower limit of quantification of 0.01 pg/μL was achieved (Non-Patent Document 2, Healey 2014).

In recent years, however, with the development of artificial nucleic acids and delivery materials, treatment at a low dose has become possible. As a result, the drug concentrations in biological samples have become lower than before, and a measurement system with higher sensitivity has been required to detect the drug at such a low concentration. Furthermore, to follow the guidelines provided by the ministerial ordinance of the Ministry of Health, Labour and Welfare, the measurement system is required to have high quantitative capability that is independent from the operator, and thus measurement by the PCR method, which is a semiquantitative method, has been unsuitable for the purpose of detecting a drug at such a low concentration.

Moreover, the conventional measurement systems have problems because, when the oligonucleotide used for the nucleic acid drug is metabolized and decomposed from the 5′ or 3′ end, the metabolite cannot be distinguished from the intact oligonucleotide which is not decomposed from the 5′ or 3′ end by metabolism (sometimes simply referred to as “intact” below) to be measured and because the accurate drug concentration cannot be measured.

To distinguish the intact oligonucleotide to be measured and a metabolite thereof and to specifically measure the nucleic acid drug having biological activity, Yu et al. have developed a hybridization-ligation ELISA assay (Non-Patent Document 4, Yu 2002). In the assay, a “template” oligonucleotide containing a complementary sequence to the oligonucleotide to be measured and a “ligation probe” are used. The “template” oligonucleotide has additional 9-mer nucleotides adjacent to the nucleotide at the 5′ end of the complementary sequence and has biotin at the 3′ end. The “ligation probe” is a 9-mer oligonucleotide having the complementary sequence to the additional 9-mer nucleotides, has phosphoric acid at the 5′ end and has digoxigenin at the 3′ end. Accordingly, when the oligonucleotide to be measured is intact, the intact oligonucleotide and the ligation probe hybridize without any gap on the template oligonucleotide. By treating the hybridization product with a ligase, the intact oligonucleotide and the ligation probe are ligated. On the other hand, when the oligonucleotide to be measured has been metabolized and lacks the nucleotide at the 3′ end side, the ligation does not occur. The ligation product is bonded to a solid phase using the biotin, and after unreacted ligation probe is removed by washing, the digoxigenin at the 3′ end of the immobilized ligation product is detected by ELISA (see Non-Patent Document 4, Yu 2002, FIG. 1).

Wei et al. disclose that S1 nuclease treatment is conducted after ligase treatment to improve the specificity of the hybridization-ligation ELISA assay (Non-Patent Document 5, Wei 2006).

The hybridization-ligation ELISA assay, however, is complicated and is unsuitable for multiplexing because an optimal ligation probe sequence has to be designed considering the sequence of the oligonucleotide to be measured.

To solve the problem of the hybridization-ligation ELISA assay, Oomori et al. have developed a method for decomposing and removing a product derived from a metabolite of the target oligonucleotide using S1 nuclease or the like and measuring a product derived from the remaining intact target oligonucleotide (Patent Document 1). In the method, the target oligonucleotide to be measured is hybridized to a complementary nucleic acid probe (3′-complementary sequence of target sequence-5′), or the target oligonucleotide to be measured having given bases such as poly(A) (a first polynucleotide) added thereto is hybridized to a complementary nucleic acid probe (3′-complementary sequence of target oligonucleotide+complementary sequence of first polynucleotide-5′), and after decomposing and removing an incomplete hybridization product using a single strand-specific nuclease such as S1 nuclease, the nucleic acid probe contained in the remaining complete hybridization product is measured.

Although the method by Oomori et al. is a method for measuring an oligonucleotide having higher sensitivity and excellent specificity and quantitative capability compared to the conventional methods such as the hybridization-ligation ELISA assay, the method is complicated due to the use of a single strand-specific nuclease such as S1 nuclease and is disadvantageous because an additional incubation time is required.

Furthermore, in “Guidance on Nonclinical Safety Studies for the Conduct of Human Clinical Trials and Marketing Authorization for Pharmaceuticals” of the Ministry of Health, Labour and Welfare, a toxicity test is required when the cross-reactivity with a metabolite exceeds 10%. As described above, although it is an important standard in developing pharmaceuticals that measurement is possible with a cross-reactivity with a metabolite of 10% or less, in the conventional methods based on hybridization such as the hybridization-ligation ELISA assay, the cross-reactivity with a metabolite is still high, and it is difficult to suppress the cross-reactivity to 10% or less stably in the detection.

To meet the severe requirements above in development of pharmaceuticals and to develop a highly versatile detection or quantification method of an oligonucleotide, the present inventors have developed a detection or quantification method for an oligonucleotide which is simple and highly sensitive and has excellent quantitative capability and capability of discriminating a metabolite, and the inventors have thus completed the invention.

### CITATION LIST

### Patent Literature

Patent Document 1: JP6718032B
Patent Document 2: WO2013/172305
Patent Document 3: JP4902674B
Patent Document 4: WO2007/037282

### Non Patent Literature

Non-Patent Document 1: Ando H, Yonenaga N, Asai T, Hatanaka K, Koide H, Tsuzuku T, Harada N, Tsukada H, Oku N. [In vivo imaging of liposomal small interfering RNA (siRNA) trafficking by positron emission tomography]. Yakugaku Zasshi. 2012; 132 (12): 1373-81. Review.
Non-Patent Document 2: Healey G D, Lockridge J A, Zinnen S, Hopkin J M, Richards I, Walker W. Development of pre-clinical models for evaluating the therapeutic potential of candidate siRNA targeting STAT6. PLOS One. 2014 Feb. 27; 9 (2): e90338.
Non-Patent Document 3: Chen C, Ridzon D A, Broomer A J, Zhou Z, Lee D H, Nguyen J T, Barbisin M, Xu N L, Mahuvakar V R, Andersen M R, Lao K Q, Livak K J, Guegler K J. Real-time quantification of microRNAs by stem-loop RT-PCR. Nucleic Acids Res. 2005 Nov. 27; 33 (20): e179.
Non-Patent Document 4: Yu R Z, Baker B, Chappell A, Geary R S, Cheung E, Levin A A. Development of an ultrasensitive noncompetitive hybridization-ligation enzyme-linked immunosorbent assay for the determination of phosphorothioate oligodeoxynucleotide in plasma. Anal Biochem. 2002 May 1; 304 (1): 19-25.
Non-Patent Document 5: Wei X, Dai G, Marcucci G, Liu Z, Hoyt D, Blum W, Chan K K. A specific picomolar hybridization-based ELISA assay for the determination of phosphorothioate oligonucleotides in plasma and cellular matrices. Pharm Res. 2006 June; 23 (6): 1251-64.

### SUMMARY OF INVENTION

### Technical Problem

The conventional signal detection methods (the hybridization method, the ligand binding assays such as the ligation method and qPCR) have a problem because an oligonucleotide in which a part of the sequence of the target oligonucleotide to be measured has been deleted, in particular an oligonucleotide in which the 5′ side or the 3′ side has been deleted (a metabolite or the like), and the intact target oligonucleotide (unchanged form) cannot be distinguished and because the metabolite and the unchanged form are both measured. Moreover, the method of Yu et al. and the method of Oomori et al., which solve the problem, require enzymatic treatment or the like of the sample and thus have a problem of the simpleness.

Here, a liquid chromatography-mass spectrometer (LC-MS) and the HPLC-UV method can make measurement while distinguishing a metabolite and the intact target oligonucleotide but have a drawback of insufficient sensitivity.

A problem to be solved by the invention is to provide a method for measuring an oligonucleotide which is simpler and more sensitive and has excellent specificity and quantitative capability compared to a conventional measurement method.

Another problem to be solved by the invention is to provide a method for measuring an oligonucleotide having excellent specificity which can distinguish the unchanged form and a metabolite and detect the unchanged form only.

### Solution to Problem

As a method for measuring an anti-drug antibody, a double antigen bridging immunoassay using a capture drug antibody and a tracer drug antibody is known (Patent Document 3). In the method, the capture drug antibody and the tracer drug antibody specifically bind to the analyte (anti-drug antibody) contained in a sample, and thus a trimer of [capture drug antibody]-[analyte]-[tracer drug antibody] is formed. By detecting the signal derived from the label contained in the tracer drug antibody, after binding the capture drug antibody to a solid phase and removing free tracer drug antibody, a signal in proportion to the analyte amount in the sample can be obtained.

The present inventors have examined a hybridization method using a capture probe and an assist probe (see Patent Document 4) (for convenience, sometimes called “CP-AP method” below) as a method for measuring a target oligonucleotide and consequently a nucleic acid drug in a sample. In the CP-AP method, a first nucleic acid probe contained in a capture probe and a second nucleic acid probe contained in an assist probe specifically hybridize to the nucleic acid drug (target oligonucleotide) contained in the sample, and thus a trimer of [capture probe]-[nucleic acid drug]-[assist probe] is formed (see FIG. 1). The present inventors have made an attempt to obtain a signal in proportion to the amount of the target oligonucleotide in the sample by removing free assist probe using the solid phase contained in the capture probe and then detecting the signal derived from the label contained in the assist probe.

The present inventors have made extensive examination to achieve measurement of an oligonucleotide, and consequently a nucleic acid drug, by the CP-AP method and thus found that by inserting a spacer between the solid phase and the nucleic acid probe contained in the capture probe, it becomes possible not only to detect the nucleic acid drug (target oligonucleotide) in a sample but also, surprisingly, to distinguish the nucleic acid drug from the metabolite thereof. The effect was particularly significant when streptavidin or avidin and biotin were used as the adapter for binding the solid phase and the nucleic acid probe in the capture probe. The present inventors have further found that the cross-reactivity with a metabolite of the nucleic acid drug can be suppressed surprisingly low using the invention.

Thus, the invention has the following configurations.

### Embodiment 1

A method for measuring a target oligonucleotide in a sample using a capture probe and an assist probe in combination by the principle of hybridization in which the target oligonucleotide retaining the full-length sequence and a metabolite thereof are distinguished,
wherein
the capture probe contains a solid phase, a first nucleic acid probe immobilized on the solid phase and a first spacer between the solid phase and the first nucleic acid probe,
the assist probe contains a tag or a label and a second nucleic acid probe linked to the tag or the label,
of the nucleotides of the first nucleic acid probe, the nucleotide which is the most proximal to the solid phase forms a base pair with the nucleotide at the 3′ end or the 5′ end of the target oligonucleotide in a preferable aspect,
the metabolite lacks one or more consecutive nucleotides including the nucleotide at the 3′ end or the 5′ end,
the first nucleic acid probe can hybridize to a part in the target oligonucleotide, the part containing the one or more nucleotides which are lacking in the metabolite,
the second nucleic acid probe can hybridize to another part in the target oligonucleotide than the part, and
the capture probe, the target oligonucleotide and the assist probe form a complex.

### Embodiment 1.2

The method according to embodiment 1, wherein the first nucleic acid probe contained in the capture probe is bonded to the first spacer through the nucleotide at the 5′ end when the target oligonucleotide in the sample is distinguished from a metabolite thereof which lacks one or more nucleotides from the 3′ end in the measurement.

### Embodiment 1.3

The method according to embodiment 1, wherein the first nucleic acid probe contained in the capture probe is bonded to the first spacer through the nucleotide at the 3′ end when the target oligonucleotide in the sample is distinguished from a metabolite thereof which lacks one or more nucleotides from the 5′ end in the measurement.

### Embodiment 2

A method for detecting a target oligonucleotide in a sample while distinguishing from a metabolite thereof which lacks one or more nucleotides from the 3′ end or the 5′ end, comprising:
(i) bringing a capture probe for capturing the target oligonucleotide and an assist probe for detecting the target oligonucleotide into contact with a sample containing the target oligonucleotide or the metabolite thereof which lacks one or more nucleotides from the 3′ end or the 5′ end and forming a complex of the capture probe, the target oligonucleotide and the assist probe,
wherein
the capture probe contains a solid phase, a first nucleic acid probe immobilized on the solid phase and a first spacer between the solid phase and the first nucleic acid probe,
the assist probe contains a tag or a label and a second nucleic acid probe linked to the tag or the label,
the sequence of the first nucleic acid probe is complementary to a partial sequence of the target oligonucleotide, wherein the partial sequence contains the one or more nucleotides which are lacking in the metabolite,
the sequence of the second nucleic acid probe is complementary to a sequence in the target oligonucleotide other than the partial sequence, and
in a preferable aspect, the first spacer is bonded to the nucleotide at an end of the first nucleic acid probe, wherein the nucleotide at the end forms a base pair with the nucleotide at the end of the target oligonucleotide which is lacking in the metabolite when the target oligonucleotide and the first nucleic acid probe hybridize; and
(ii) detecting the target oligonucleotide in the sample by detecting the complex.

### Embodiment 2.1

The method according to embodiment 2, wherein when the target oligonucleotide in the sample is distinguished from a metabolite thereof which lacks one or more nucleotides from the 3′ end in the detection, the first nucleic acid probe is bonded to the first spacer through the nucleotide at the 5′ end, and the sequence of the first nucleic acid probe is complementary to a sequence in the target oligonucleotide containing the 3′ end.

### Embodiment 2.2

The method according to embodiment 2, wherein when the target oligonucleotide in the sample is distinguished from a metabolite thereof which lacks one or more nucleotides from the 5′ end in the detection, the first nucleic acid probe is bonded to the first spacer through the nucleotide at the 3′ end, and the sequence of the first nucleic acid probe is complementary to a sequence in the target oligonucleotide containing the 5′ end.

### Embodiment 3

A method for detecting a target oligonucleotide in a sample, comprising:
(i) bringing a capture probe for capturing the target oligonucleotide and an assist probe for detecting the target oligonucleotide into contact with a sample and forming a complex of the capture probe, the target oligonucleotide and the assist probe,
wherein
the capture probe contains a solid phase, a first nucleic acid probe immobilized on the solid phase and a first spacer between the solid phase and the first nucleic acid probe,
the assist probe contains a tag or a label and a second nucleic acid probe linked to the tag or the label,
the sequence of the first nucleic acid probe is complementary to a partial sequence of the target oligonucleotide including the nucleotide at an end of the target oligonucleotide,
the sequence of the second nucleic acid probe is complementary to a sequence in the target oligonucleotide other than the partial sequence, and
in a preferable aspect, the first spacer is bonded to the nucleotide at an end of the first nucleic acid probe, wherein the nucleotide at the end of the first nucleic acid probe forms a base pair with the nucleotide at the end of the target oligonucleotide when the target oligonucleotide and the first nucleic acid probe hybridize; and
(ii) detecting the target oligonucleotide in the sample by detecting the complex.

### Embodiment 3.1

The method according to embodiment 3, wherein the sequence of the first nucleic acid probe is complementary to a partial sequence at the 3′ side of the target oligonucleotide including the nucleotide at the 3′ end of the target oligonucleotide, the sequence of the second nucleic acid probe is complementary to a sequence in the target oligonucleotide other than the partial sequence at the 3′ side, and the first spacer is bonded to the nucleotide at the 5′ end of the first nucleic acid probe.

### Embodiment 3.2

The method according to embodiment 3, wherein the sequence of the first nucleic acid probe is complementary to a partial sequence at the 5′ side of the target oligonucleotide including the nucleotide at the 5′ end of the target oligonucleotide, the sequence of the second nucleic acid probe is complementary to a sequence in the target oligonucleotide other than the partial sequence at the 5′ side, and the first spacer is bonded to the nucleotide at the 3′ end of the first nucleic acid probe.

### Embodiment 3.3

The method according to any of embodiments 1 to 3.2, wherein the first nucleic acid probe contained in the capture probe is 5-nucleotide length, 6-nucleotide length, 7-nucleotide length, 8-nucleotide length, 9-nucleotide length, 10-nucleotide length or 11-nucleotide length.

### Embodiment 3.4

The method according to any of embodiments 1 to 3.3, wherein the second nucleic acid probe contained in the assist probe is 4-nucleotide length, 5-nucleotide length, 6-nucleotide length, 7-nucleotide length, 8-nucleotide length, 9-nucleotide length, 10-nucleotide length, 11-nucleotide length, 12-nucleotide length, 13-nucleotide length, 14-nucleotide length, 15-nucleotide length, 16-nucleotide length, 17-nucleotide length, 18-nucleotide length, 19-nucleotide length, 20-nucleotide length, 21-nucleotide length, 22-nucleotide length, 23-nucleotide length, 24-nucleotide length or 25-nucleotide length: wherein in an aspect, when the first nucleic acid probe contained in the capture probe is 11-nucleotide length, the second nucleic acid probe contained in the assist probe is not 4-nucleotide length.

### Embodiment 4

The method according to any of embodiments 1 to 3.4, wherein the capture probe contains an adapter between the solid phase and the first spacer.

### Embodiment 5

The method according to embodiment 4, wherein the capture probe contains a second spacer between the solid phase and the adapter.

### Embodiment 6

The method according to embodiment 4 or 5, wherein the adapter is streptavidin or avidin and biotin.

### Embodiment 7

The method according to embodiment 6, wherein the binding of the streptavidin or the avidin and the biotin occurs after the formation of the complex of the capture probe, the target oligonucleotide and the assist probe.

### Embodiment 8

The method according to any of embodiments 1 to 7, wherein the first spacer is a 5-mer to 90-mer oligonucleotide.

### Embodiment 8.1

The method according to any of embodiments 1 to 8, further comprising:
(i) adding to the complex a pair of self-assembly signal amplification probes having complementary base sequence regions that can hybridize to each other and forming a probe polymer bonded to the complex; and
(ii) detecting the probe polymer;
wherein the assist probe contains a tag having a base sequence complementary to a part of or the whole of one signal amplification probe of the pair of self-assembly signal amplification probes.

### Embodiment 8.2

The method according to embodiment 8.1, wherein at least one of the pair of self-assembly signal amplification probes contains a poly T sequence.

### Embodiment 8.3

The method according to embodiment 8.1 or 8.2, wherein at least one of the pair of self-assembly signal amplification probes is labeled with a labeling substance.

### Embodiment 8.4

The method according to any of embodiments 8.1 to 8.3,
wherein
the pair of self-assembly signal amplification probes contains a first signal amplification probe and a second signal amplification probe,
the first signal amplification probe is a nucleic acid probe containing three or more nucleic acid regions and containing at least a nucleic acid region X, a nucleic acid region Y and a nucleic acid region Z or a nucleic acid region Z containing a poly T sequence in this order from the 5′ end side, and
the second signal amplification probe is a nucleic acid probe containing three or more nucleic acid regions and containing at least a nucleic acid region X′ which is complementary to the nucleic acid region X, a nucleic acid region Y′ which is complementary to the nucleic acid region Y and a nucleic acid region Z′ which is complementary to the nucleic acid region Z or a nucleic acid region Z′ containing a poly A sequence in this order from the 5′ end side.

### Embodiment 9

A detection kit for use in detecting a target oligonucleotide, comprising a capture probe, an assist probe and a pair of signal amplification probes which has complementary base sequence regions that can hybridize to each other and which is capable of forming a probe polymer through self-assembly,
wherein
the capture probe contains a solid phase, a first nucleic acid probe immobilized on the solid phase and a first spacer between the solid phase and the first nucleic acid probe,
the assist probe contains a tag having a base sequence which is complementary to a part of or the whole of one signal amplification probe of the pair of signal amplification probes and a second nucleic acid probe linked to the tag,
the sequence of the first nucleic acid probe is complementary to a partial sequence of the target oligonucleotide including the nucleotide at an end of the target oligonucleotide, and
the sequence of the second nucleic acid probe is complementary to a sequence in the target oligonucleotide other than the partial sequence,
in a preferable aspect, the first spacer is bonded to the nucleotide at an end of the first nucleic acid probe, wherein the nucleotide at the end of the first nucleic acid probe forms a base pair with the nucleotide at the end of the target oligonucleotide when the target oligonucleotide and the first nucleic acid probe hybridize.

### Embodiment 10

The detection kit according to embodiment 9, wherein the capture probe contains an adapter between the solid phase and the first spacer.

### Embodiment 11

The detection kit according to embodiment 10, wherein the capture probe contains a second spacer between the solid phase and the adapter.

### Embodiment 12

The detection kit according to embodiment 10 or 11, wherein the adapter is streptavidin or avidin and biotin.

### Embodiment 13

The detection kit according to embodiment 12, wherein the binding of the streptavidin or the avidin and the biotin occurs after the formation of the complex of the capture probe, the target oligonucleotide and the assist probe.

### Embodiment 14

The detection kit according to any of embodiments 9 to 13, wherein the first spacer is a 5-mer to 90-mer oligonucleotide.

### Embodiment 14.1

The detection kit according to embodiment 9 or 10, wherein the detection kit is for measuring the target oligonucleotide in a sample while distinguishing from a metabolite thereof which lacks one or more nucleotides from the 3′ end, and wherein the first nucleic acid probe contained in the capture probe is bonded to the first spacer through the nucleotide at the 5′ end.

### Embodiment 14.2

The detection kit according to embodiment 9 or 10, wherein the detection kit is for measuring the target oligonucleotide in a sample while distinguishing from a metabolite thereof which lacks one or more nucleotides from the 5′ end, and wherein the first nucleic acid probe contained in the capture probe is bonded to the first spacer through the nucleotide at the 3′ end.

### Embodiment 15

The detection kit according to any of embodiments 9 to 14.2, wherein at least one of the pair of signal amplification probes is labeled with a labeling substance.

### Embodiment 16

The detection kit according to any of embodiments 9 to 15,
wherein
the pair of signal amplification probes contains a first signal amplification probe and a second signal amplification probe,
the first signal amplification probe is a nucleic acid probe containing at least a nucleic acid region X, a nucleic acid region Y and a nucleic acid region Z or a nucleic acid region Z containing a poly T sequence in this order from the 5′ end side, and
the second signal amplification probe is a nucleic acid probe containing at least a nucleic acid region X′ which is complementary to the nucleic acid region X, a nucleic acid region Y′ which is complementary to the nucleic acid region Y and a nucleic acid region Z′ which is complementary to the nucleic acid region Z or a nucleic acid region Z′ containing a poly A sequence in this order from the 5′ end side.

### Advantageous Effects of Invention

By the invention, detection or quantification of an oligonucleotide which is simple and highly sensitive and has excellent specificity and quantitative capability can be conducted by a step of hybridization of probes only without using any enzyme or the like. Moreover, an oligonucleotide detection method or an oligonucleotide quantification method having excellent specificity and quantitative capability which can discriminate an oligonucleotide metabolite in which the 5′ side or the 3′ side of the target oligonucleotide to be measured has been deleted and the unchanged form, which has little cross-reactivity with the metabolite, and which can detect only the unchanged form, can be provided.

### BRIEF DESCRIPTION OF DRAWINGS

FIG. 1 A figure showing the principle of the hybridization method using a capture probe and an assist probe (CP-AP method).

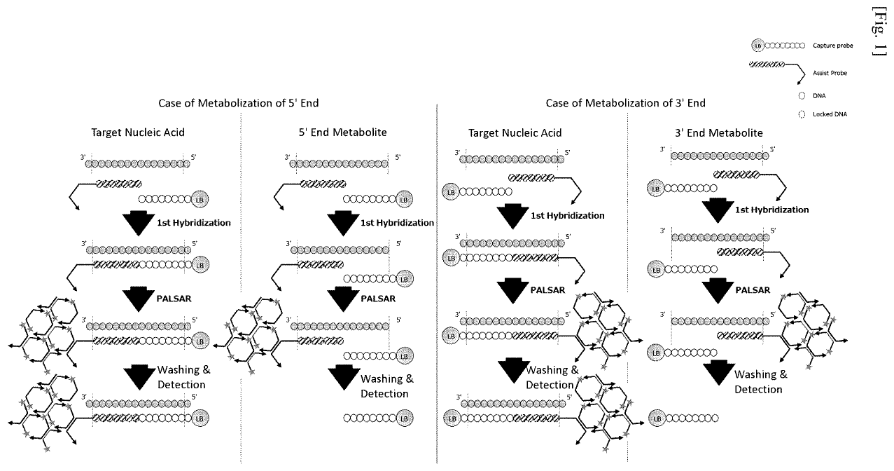

FIG. 2 A figure showing the structures of elements of LNA and DNA.

### DESCRIPTION OF EMBODIMENTS

### (Sample)

A “sample” used in the method of the invention is a body fluid such as whole blood, serum, plasma, lymph fluid, urine, saliva, tear fluid, sweat, gastric juice, pancreatic fluid, bile, pleural effusion, intraarticular fluid, cerebrospinal fluid, spinal fluid and bone marrow aspirate, a tissue such as liver, kidney, lung and heart or the like of a human, a monkey, a dog, a pig, a rat, a guinea pig or a mouse. The sample is preferably whole blood, serum, plasma or urine of a human, a monkey, a dog, a pig, a rat, a guinea pig or a mouse, preferably of a human. Further preferably, the sample is whole blood, serum, plasma or urine of a human, a monkey, a dog, a pig, a rat, a guinea pig or a mouse, preferably of a human, to which a medicine containing a target oligonucleotide has been administered.

### (Target Oligonucleotide)

In the present specification, the term “target oligonucleotide” means an intact oligonucleotide (intact target oligonucleotide/unchanged form) to be measured. That is, the term “target oligonucleotide” does not include the metabolites from which it should be distinguished. In the present specification, as long as a specific hybrid with a nucleic acid probe can be formed, the term “target oligonucleotide” may be DNA or RNA, may be single-stranded or double-stranded or may be chemically modified. The chemical modification is phosphorothioate modification, 2′-F modification, 2′-O-methyl (2′-OMe) modification, 2′-O-methoxyethyl (2′-MOE) modification, morpholino modification, LNA modification, BNACOC modification, BNANC modification, ENA modification, cEt BNA modification or the like. When the target oligonucleotide is double-stranded, the target oligonucleotide is used in the invention after converting into a single strand. The nucleotide length of the target oligonucleotide is not limited but is preferably 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer or 30-mer.

### (Capture Probe)

A “capture probe” used in the invention is a probe for capturing the target oligonucleotide and contains a nucleic acid probe, a first spacer which is adjacent to, immobilized on, bonded to or linked to the nucleotide at the 3′ end or the 5′ end of the nucleic acid probe and a solid phase which is adjacent to, immobilized on, bonded to or linked to the first spacer.

### (Assist Probe)

An “assist probe” used in the invention is a probe for detecting the target oligonucleotide and contains a nucleic acid probe and a tag or a label adjacent to the nucleotide at the 5′ end or the 3′ end of the nucleic acid probe.

### (Nucleic Acid Probes Contained in Capture Probe and Assist Probe-Regarding Constituent Nucleotides)

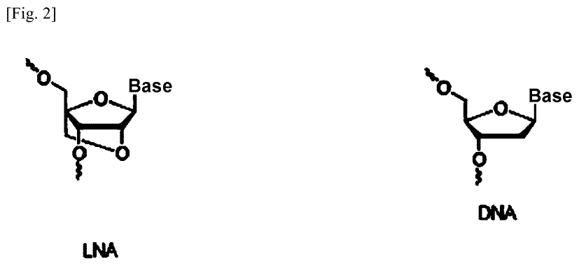

The nucleic acid probes contained in the capture probe and the assist probe are composed of deoxyribonucleotides or ribonucleotides but, in an aspect of the invention, each independently contain zero, one, two, three, four, five, six, seven, eight, nine, ten or 11 locked nucleic acids (LNAs) (FIG. 2). When the nucleic acid probes have a nucleotide length of 5-mer, the nucleic acid probes preferably contain five locked nucleic acids (LNAs), and when the nucleic acid probes have a nucleotide length of 6-mer to 11-mer, the nucleic acid probes preferably contain zero, one, two, three, four, five, six, seven, eight, nine, ten or 11 locked nucleic acids (LNAs).

### (Nucleic Acid Probes-Regarding Nucleotide Lengths)

The nucleic acid probe contained in the capture probe has a nucleotide length of 5-mer, 6-mer, 7-mer, 8-mer, 9-mer, 10-mer or 11-mer in an aspect. In the aspect, a target oligonucleotide retaining the full-length sequence in a sample was able to be actually measured while distinguishing from a metabolite thereof which lacked one or more nucleotides from the 3′ end or the 5′ end.

The nucleic acid probe contained in the assist probe may be able to bind to a region other than the nucleic acid probe region contained in the capture probe and has a nucleotide length of 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer or 25-mer in an aspect. In the aspect, a target oligonucleotide retaining the full-length sequence in a sample was able to be actually measured while distinguishing from a metabolite thereof which lacked one or more nucleotides from the 3′ end or the 5′ end. The nucleic acid probe contained in the assist probe has a nucleotide length of 4-mer, 5-mer, 6-mer, 7-mer, 8-mer, 9-mer, 10-mer, 11-mer, 12-mer, 13-mer, 14-mer, 15-mer or 16-mer in another aspect. In the aspect, a target oligonucleotide retaining the full-length sequence in a sample was able to be actually measured while distinguishing from a metabolite thereof which lacked one or more nucleotides from the 3′ end or the 5′ end.

In this regard, however, in an aspect, the aspect in which the nucleic acid probe contained in the capture probe has a nucleotide length of 11-mer and in which the nucleic acid probe contained in the assist probe has a nucleotide length of 4-mer is not included in the invention. In an aspect, the aspect in which the nucleic acid probe contained in the capture probe has a nucleotide length of 10-mer and in which the nucleic acid probe contained in the assist probe has a nucleotide length of 4-mer is included in the invention. In another aspect, the aspect in which the nucleic acid probe contained in the capture probe has a nucleotide length of 10-mer and in which the nucleic acid probe contained in the assist probe has a nucleotide length of 4-mer is not included in the invention.

### (Contact)

In the present specification, the term bringing into “contact” or the step of bringing into “contact” means that a substance and another substance are placed close to each other so that the substances can form a chemical bond such as a covalent bond, an ionic bond, a metal bond and a noncovalent bond. In an aspect of the invention, bringing a substance and another substance “into contact” means that a solution containing the substance and a solution containing the other substance are mixed. In the invention, by bringing the capture probe, the target oligonucleotide and the assist probe into contact with each other, a complex thereof is formed. In an aspect, the step of bringing the capture probe and the assist probe into contact with the sample is conducted by incubating a mixture containing the sample, the capture probe, and the assist probe and the target oligonucleotide at a temperature which is +2° C. to −10° C., +1° C. to −9° C., 0° C. to −8° C., −1° C. to −7° C., −2° C. to −6° C. or −3° C. to −5° C. or is +10° C., +9° C., +8° C., +7° C., +6° C., +5° C., +4° C., +3° C., +2° C., +1° C., 0° C., −1° C., −2° C., −3° C., −4° C., −5° C., −6° C., −7° C., −8° C., −9° C. or −10° C. compared to the melting temperature (Tm) of the nucleic acid probe contained in the capture probe for a certain period. For example, when the Tm is 50° C., +2° C. to −10° C. compared to the Tm means 52° C. to 40° C. In an aspect, the incubation period is 10 seconds to four minutes, 20 seconds to three minutes or 30 seconds to two minutes or is 10 seconds, 20 seconds, 30 seconds, 40 seconds, 50 seconds, 60 seconds, 70 seconds, 80 seconds, 90 seconds, 100 seconds, 110 seconds, 120 seconds, 130 seconds, 140 seconds, 150 seconds, 160 seconds, 170 seconds or 180 seconds.

### (Capture)

In the invention, that a capture probe “captures” a target oligonucleotide principally means that the nucleic acid probe contained in the capture probe and the target oligonucleotide hybridize. In an aspect, that the capture probe “captures” the target oligonucleotide means that the target oligonucleotide binds indirectly, through the nucleic acid probe and the spacer contained in the capture probe, to the solid phase contained in the capture probe or to the solid phase to which the adapter is attached. In the invention, because the capture probe directly captures the target oligonucleotide and further indirectly captures the assist probe through the target oligonucleotide, a signal in proportion to the amount of the target oligonucleotide in the sample can be obtained from the assist probe.

### (Hybridization/To Hybridize)

In the present specification, that a nucleic acid probe contained in a capture probe or an assist probe hybridizes to a target oligonucleotide means that a single-stranded nucleic acid probe having a sequence that is complementary to a part of a particular base sequence binds to a single-stranded target oligonucleotide having the particular base sequence by forming base pairs and that a double-stranded nucleic acid molecule is formed.

### (Complex)

In the present specification, that a “complex” of the capture probe, the target oligonucleotide, and the assist probe is formed means to form a trimer in which the nucleic acid probe contained in the capture probe and a part of the target oligonucleotide specifically hybridize and in which the nucleic acid probe contained in the assist probe and another part of the target oligonucleotide specifically hybridize. Here, that the nucleic acid probe and a part of the target oligonucleotide specifically hybridize means that all the bases contained in the nucleic acid probe excluding the spacer and the tag form pairs with the bases of the target oligonucleotide. In an aspect, all the bases contained in the target oligonucleotide form pairs with the bases of the nucleic acid probe contained in the capture probe or the bases of the nucleic acid probe contained in the assist probe.

### (Removal)

During the detection of the complex of the capture probe, the target oligonucleotide and the assist probe, free assist probe is preferably removed when the signal derived from the free assist probe prevents the detection. For example, by washing the solid phase contained in the capture probe or the solid phase contained in the capture probe which is bonded through an adapter, the free assist probe can be removed. To wash the solid phase, the reaction solution in which the solid phase is suspended may be subjected to a centrifuge or filtered to separate the liquid phase of the reaction solution. Moreover, when the solid phase is magnetic, the solid phase can be recovered using a magnet. The solid phase may be washed multiple times according to the need.

### (Detection)

To “detect” the target oligonucleotide, the tag or the label contained in the assist probe or the label bonded through the tag can be used. Moreover, when the solid phase contained in the capture probe can emit a signal such as fluorescence, the signal can also be used. The signal from the label or the solid phase may be any signal as long as the signal can be detected physically or chemically, but a signal which can be detected optically is preferable to achieve high throughput.

### (Self-Assembly: PALSAR Method)

Self-assembly means the state in which a plurality of the first signal amplification probe molecules form a probe polymer through hybridization to the second signal amplification probe and the state in which a plurality of the second signal amplification probe molecules form a probe polymer through hybridization to the first signal amplification probe.

### (Pair of Self-Assembly Signal Amplification Probes)

A pair of “self-assembly” signal amplification probes used in the method of the invention refer to oligonucleotides in which the first signal amplification probe and the second signal amplification probe have complementary base sequence regions that can hybridize to each other and which can form a probe polymer through self-assembly reaction. Here, “hybridizable” means that the complementary base sequence regions are completely complementary in an aspect.

The pair of self-assembly probes can be labeled with a labeling substance for detection in advance. Preferably, at least one of the first and second signal amplification probes is labeled with a labeling substance. Such a labeling substance may be a radioisotope, biotin, digoxigenin, a fluorescent substance, a luminescent substance, a dye or the like. Specific examples include radioisotopes such as ¹²⁵I and ³²P, digoxigenin, luminescent/chromogenic substances such as acridinium esters, alkaline phosphatase for using a luminescent substance such as dioxetane or a fluorescent substance such as 4-methylumbelliferyl phosphate, biotin for using a fluorescent/luminescent/chromogenic substance bonded to avidin or the like and the like. Moreover, a donor fluorescent dye and an acceptor fluorescent dye for using fluorescence resonance energy transfer (FRET) can be added to detect the target oligonucleotide.

In an aspect, the labeling substance is biotin, and the oligonucleotide is labeled by biotinylating the 5′ end or the 3′ end. When the labeling substance is biotin, the substance which specifically binds to the labeling substance is streptavidin or avidin. In an aspect, the labeling substance is not biotin, and the substance which specifically binds to the labeling substance is not streptavidin or avidin.

In some cases, detection is conducted by bringing the pair of self-assembly probes composed of the first and second signal amplification probes into contact with a complex of the invention containing the hybridization product of, the target oligonucleotide, the capture probe, and the assist probe, and thus, binding the probe polymer composed of the first and second signal amplification probes to the complex.

In an aspect, the assist probe used above contains a tag which can bind to one of the pair of self-assembly probes composed of the first and second signal amplification probes and has a role of assisting binding of the target oligonucleotide and the probe polymer. A first aspect of the assist probe is a probe containing a tag having a complementary sequence to the entire sequence or a partial sequence of at least one of the first and second oligonucleotides and a complementary sequence to a partial sequence of the target oligonucleotide.

### (Solid Phase)

In the present specification, examples of the term “solid phase” include insoluble microparticles, microbeads, fluorescent microparticles, magnetic particles, a microplate, a microarray, a microscope slide, a substrate such as an electroconductive substrate and the like.

The “solid phase” is fluorescent microparticles in an aspect of the invention, fluorescent beads in another aspect or beads having a fluorescent substance on the surface in another aspect. The “beads having a fluorescent substance on the surface” used in the invention are not particularly limited as long as the beads have a fluorescent substance, and for example, MicroPlex™ Microspheres of Luminex can be preferably used. One kind of beads may be used, or many kinds of beads can also be used. When multiple kinds of color-coded beads are used, the method for quantifying an oligonucleotide of the invention can also be easily multiplexed.

The “solid phase” is a microplate in an aspect of the invention. The material of the microplate used in the invention may be polystyrene, polypropylene, polycarbonate or a cyclic olefin copolymer but is not limited thereto. In an aspect of the invention, the microplate is a coated plate such as a biotin-coated plate, a protein A-, G-, A/G- and/or L-coated plate, an anti-GST antibody-coated plate, a glutathione-, nickel- and/or copper-coated plate, an amine- and/or sulfhydryl-bonded plate, a carboxylated plate and a streptavidin-coated plate.

In an aspect, the solid phase is not insoluble microparticles, not microbeads, not fluorescent microparticles, not magnetic particles, not a microplate, not a microarray, not a microscope slide or not a substrate such as an electroconductive substrate and the like.

### (Adapter)

Examples of an “adapter” used in the invention include biotin, streptavidin or avidin, a combination thereof, an antigen, an antibody and a combination thereof, and the adapter is preferably biotin, streptavidin or avidin, a combination thereof or the like. In an aspect, the adapter is not a nucleic acid such as an oligonucleotide and a nucleotide, is none of biotin, streptavidin, avidin, a combination thereof, an antigen, an antibody and a combination thereof and is not a compound having an amino group or a carboxy group such as spacers including Spacer 9, Spacer 12, Spacer 18, Spacer C3 and the like or the like. In another aspect, the adapter does not contain any nucleic acid such as an oligonucleotide and a nucleotide. Furthermore, in an aspect, streptavidin or avidin is directly immobilized on the solid phase. Moreover, in another aspect, streptavidin or avidin is not directly immobilized on the solid phase. For example, in the other aspect, streptavidin or avidin is immobilized on the solid phase through a (second) spacer.

### (Spacer)

Examples of a “spacer” used in the invention include a nucleic acid such as an oligonucleotide and a nucleotide, a compound having an amino group or a carboxy group such as spacers including Spacer 9, Spacer 12, Spacer 18, Spacer C3 and the like and the like, and the spacer is preferably 5′-Amino-Modifier C12 (12-(4-monomethoxytritylamino) dodecyl-1-[(2-cyanoethyl)-(N,N-diisopropyl)]-phosphoramidite) or the like. For example, in an aspect in which beads have a carboxy group on the surface and in which a nucleic acid probe to which a compound having an amino group is added is bonded to the carboxy group on the bead surface through the amino group, the compound having the amino group is an example of the spacer. In an aspect, the spacer is not a nucleic acid such as an oligonucleotide and a nucleotide, is not biotin and is not a compound having an amino group or a carboxy group such as spacers including Spacer 9, Spacer 12, Spacer 18, Spacer C3 and the like or the like. In another aspect, the spacer does not contain any nucleic acid such as an oligonucleotide and a nucleotide. Furthermore, in an aspect, the first spacer is directly immobilized on the solid phase. Moreover, in another aspect, the first spacer is not directly immobilized on the solid phase. For example, in the other aspect, the first spacer is immobilized on the solid phase through biotin, streptavidin or avidin or a combination thereof.

When the spacer is an oligonucleotide, the nucleotide length of the oligonucleotide is 4-mer or more and 130-mer or less, 5-mer or more and 90-mer or less, 7-mer or more and 50-mer or less, 10-mer or more and 40-mer or less or 15-mer or more and 30-mer or less or is 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, 40-mer, 41-mer, 42-mer, 43-mer, 44-mer, 45-mer, 46-mer, 47-mer, 48-mer, 49-mer, 50-mer, 51-mer, 52-mer, 53-mer, 54-mer, 55-mer, 56-mer, 57-mer, 58-mer, 59-mer, 60-mer, 61-mer, 62-mer, 63-mer, 64-mer, 65-mer, 66-mer, 67-mer, 68-mer, 69-mer, 70-mer, 71-mer, 72-mer, 73-mer, 74-mer, 75-mer, 76-mer, 77-mer, 78-mer, 79-mer, 80-mer, 81-mer, 82-mer, 83-mer, 84-mer, 85-mer, 86-mer, 87-mer, 88-mer, 89-mer, 90-mer, 91-mer, 92-mer, 93-mer, 94-mer, 95-mer, 96-mer, 97-mer, 98-mer, 99-mer, 100-mer, 101-mer, 102-mer, 103-mer, 104-mer, 105-mer, 106-mer, 107-mer, 108-mer, 109-mer, 110-mer, 111-mer, 112-mer, 113-mer, 114-mer, 115-mer, 116-mer, 117-mer, 118-mer, 119-mer, 120-mer, 121-mer, 122-mer, 123-mer, 124-mer, 125-mer, 126-mer, 127-mer, 128-mer, 129-mer or 130-mer.

### (Tag or Label)

A “tag” contained in the assist probe may be a poly A sequence, a poly T sequence, a poly U sequence, a poly (T/U) sequence, a poly G sequence, a poly C sequence or a nucleic acid which contains any specific sequence or which is composed thereof. The nucleotide length of the nucleic acid tag is 5-mer or more and 115-mer or less, 10-mer or more and 110-mer or less, 15-mer or more and 105-mer or less, 20-mer or more and 100-mer or less, 25-mer or more and 95-mer or less, 30-mer or more and 90-mer or less, 35-mer or more and 85-mer or less, 40-mer or more and 80-mer or less, 45-mer or more and 75-mer or less, 50-mer or more and 70-mer or less or 55-mer or more and 65-mer or less or is 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, 40-mer, 41-mer, 42-mer, 43-mer, 44-mer, 45-mer, 46-mer, 47-mer, 48-mer, 49-mer, 50-mer, 51-mer, 52-mer, 53-mer, 54-mer, 55-mer, 56-mer, 57-mer, 58-mer, 59-mer, 60-mer, 61-mer, 62-mer, 63-mer, 64-mer, 65-mer, 66-mer, 67-mer, 68-mer, 69-mer, 70-mer, 71-mer, 72-mer, 73-mer, 74-mer, 75-mer, 76-mer, 77-mer, 78-mer, 79-mer, 80-mer, 81-mer, 82-mer, 83-mer, 84-mer, 85-mer, 86-mer, 87-mer, 88-mer, 89-mer, 90-mer, 91-mer, 92-mer, 93-mer, 94-mer, 95-mer, 96-mer, 97-mer, 98-mer, 99-mer, 100-mer, 101-mer, 102-mer, 103-mer, 104-mer, 105-mer, 106-mer, 107-mer, 108-mer, 109-mer, 110-mer, 111-mer, 112-mer, 113-mer, 114-mer or 115-mer. In an aspect, the tag or the label does not contain any nucleic acid such as an oligonucleotide and a nucleotide. The “label” contained in the assist probe may be a radioisotope, biotin, digoxigenin, a fluorescent substance, a luminescent substance, a dye or the like. Specific examples include radioisotopes such as ¹²⁵I and ³²P, digoxigenin, luminescent/chromogenic substances such as acridinium esters, alkaline phosphatase for using a luminescent substance such as dioxetane or a fluorescent substance such as 4-methylumbelliferyl phosphate, biotin for using a fluorescent/luminescent/chromogenic substance bonded to avidin or the like and the like. Moreover, a donor fluorescent dye and an acceptor fluorescent dye for using fluorescence resonance energy transfer (FRET) can be added to detect the target oligonucleotide. In an aspect, the label may be contained in another nucleic acid molecule which hybridizes to the nucleic acid tag contained in the assist probe. In an aspect, the “label” contained in the assist probe is not a radioisotope, biotin, digoxigenin, a fluorescent substance, a luminescent substance, a dye or the like. In particular, when biotin, streptavidin or avidin and a combination thereof are used as the adapter, the “label” contained in the assist probe is not biotin in an aspect.

### (Adjacent)

That a first spacer, a tag or a label is “adjacent” to, “immobilized” on, “bonded” to or “linked” to the nucleotide at the 5′ end or the 3′ end of a nucleic acid probe principally means that the first spacer, the tag or the label is directly bonded to the nucleotide. For example, when the first spacer, the tag or the label is bonded to the nucleotide through any molecule, the molecule itself can be considered as the first spacer, the tag or the label, or the molecule itself can be considered to constitute a part of the first spacer, the tag or the label. The solid phase may be bonded to the first spacer and the nucleic acid probe through an adapter.

(Nucleic Acid Probes-Regarding Relation with Target Oligonucleotide and Metabolite Thereof)

In the invention, a metabolite refers to an oligonucleotide which lacks at least one or more nucleotides from the 3′ end and/or the 5′ end in the target oligonucleotide.

In an aspect of the invention, the metabolite of a target oligonucleotide lacks one or more nucleotides from the 3′ end. The sequence of the “nucleic acid probe” contained in a capture probe is complementary to a partial sequence of the target oligonucleotide including the nucleotide at the 3′ end of the target oligonucleotide, and the sequence of the “nucleic acid probe” contained in the assist probe is complementary to a sequence in the target oligonucleotide other than the partial sequence. In the aspect, the first spacer contained in the capture probe is adjacent to the nucleotide at the 5′ end of the nucleic acid probe contained in the capture probe, and the tag or the label contained in the assist probe is adjacent to the nucleotide at the 3′ end of the nucleic acid probe contained in the assist probe.

In another aspect of the invention, the metabolite of the target oligonucleotide lacks one or more nucleotides from the 5′ end. The sequence of the “nucleic acid probe” contained in the capture probe is complementary to a partial sequence of the target oligonucleotide including the nucleotide at the 5′ end of the target oligonucleotide, and the sequence of the “nucleic acid probe” contained in the assist probe is complementary to a sequence in the target oligonucleotide other than the partial sequence. In the aspect, the first spacer contained in the capture probe is adjacent to the nucleotide at the 3′ end of the nucleic acid probe contained in the capture probe, and the tag or the label contained in the assist probe is adjacent to the nucleotide at the 5′ end of the nucleic acid probe contained in the assist probe.

Here, in the present specification, for convenience, the “nucleic acid probe” contained in the capture probe is sometimes called the “first nucleic acid probe”, and the “nucleic acid probe” contained in the assist probe is sometimes called the “second nucleic acid probe”.

The first nucleic acid probe contained in the capture probe and the second nucleic acid probe contained in the assist probe may be adjacent to each other (without any gap) or do not have to be adjacent to each other (with a gap of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 nucleotides) when the probes hybridize to the target oligonucleotide. In an aspect of the invention, the first nucleic acid probe contained in the capture probe and the second nucleic acid probe contained in the assist probe are not adjacent but have a gap of one to 21 nucleotides, one to 16 nucleotides, one to 11 nucleotides or one to seven nucleotides when the probes hybridize to the target oligonucleotide.

Even when the first nucleic acid probe contained in the capture probe and the second nucleic acid probe contained in the assist probe were not adjacent to each other (with gaps of three, six, nine, 11 and 12 nucleotides) when the probes hybridized to the target oligonucleotide, the target oligonucleotide retaining the full-length sequence in the sample was able to be actually measured while distinguishing from a metabolite which lacked one or more nucleotides from the 3′ end or the 5′ end. A person skilled in the art would understand that the presence of a gap of one or two, one to three, one to four, one to five, one to six, one to seven, one to eight, one to nine, one to ten, one to eleven, one to twelve, one to thirteen, one to fourteen, one to fifteen, one to sixteen, one to seventeen, one to eighteen, one to nineteen, one to twenty, or one to twenty-one nucleotides does not cause any real problem.

As another aspect in which the first nucleic acid probe contained in the capture probe and the second nucleic acid probe contained in the assist probe have a gap, a blocking probe which is adjacent to each of the first nucleic acid probe contained in the capture probe and the second nucleic acid probe contained in the assist probe and which hybridizes to the gap site of the target oligonucleotide may be used. One skilled in the art would understand that a gap may be included between the first nucleic acid probe and the blocking probe and/or between the second nucleic acid probe and the blocking probe in the aspect.

### (Complementary—Regarding Capture Probe)

That the nucleic acid probe contained in a capture probe is “complementary” to a sequence at the 3′ side (5′ side) of the target oligonucleotide preferably means that the sequence of the nucleic acid probe is completely complementary to a consecutive nucleotide sequence including the nucleotide at the 3′ end (5′ end) of the target oligonucleotide. The length of the completely complementary sequence is preferably the same as the nucleotide length of the nucleic acid probe contained in the capture probe. In this regard, however, in an aspect, the nucleic acid probe can have an additional nucleotide at the 5′ end (3′ end) in addition to the completely complementary part to the sequence at the 3′ side (5′ side) of the target oligonucleotide. It is easily understood that the additional nucleotide does not have any base to form a pair or a mismatch with in the target oligonucleotide. The additional nucleotide can also be considered to constitute a part of or the whole of the solid phase, the adapter or the spacer adjacent to the nucleotide at the 5′ end (3′ end) of the nucleic acid probe. Moreover, in an aspect, one skilled in the art would understand that an artificial mutation can be introduced into the sequence of the nucleic acid probe on the condition that the nucleic acid probe can bind to the target oligonucleotide preferentially over the metabolite. The contents in the brackets are appropriately read.

### (Complementary—Regarding Assist Probe)

That the nucleic acid probe contained in an assist probe is “complementary” to a sequence at the 5′ side (3′ side) of the target oligonucleotide preferably means that the sequence of the nucleic acid probe is completely complementary to a consecutive nucleotide sequence including the nucleotide at the 5′ end (3′ end) of the target oligonucleotide. The length of the completely complementary sequence is preferably the same as the nucleotide length of the nucleic acid probe contained in the assist probe. In this regard, however, in an aspect, the nucleic acid probe can have an additional nucleotide at the 3′ end (5′ end) in addition to the completely complementary part to the sequence at the 5′ side (3′ side) of the target oligonucleotide. It is easily understood that the additional nucleotide does not have any base to form a pair or a mismatch with in the target oligonucleotide. The additional nucleotide can also be considered to constitute a part of or the whole of the tag adjacent to the nucleotide at the 3′ end (5′ end) of the nucleic acid probe. Moreover, in an aspect, one skilled in the art would understand that an artificial mutation can be introduced into the sequence of the nucleic acid probe. The contents in the brackets are appropriately read.

### EXAMPLES

### [Example 1] Test of Effect of Inserting Spacer Between Solid Phase and Nucleic Acid Probe Contained in Capture Probe

### 1. Materials and Methods

### (1) Target Nucleic Acid

PT1 was used as the target nucleic acid to be measured. As a metabolite model nucleic acid of the target nucleic acid, a nucleic acid PT1-3n-1 which lacked one base at the 3′ end was used. The synthesis of the nucleic acids was outsourced at NIHON GENE RESEARCH LABORATORIES Inc. (HPLC purification grade). The target nucleic acid above is fully phosphorothioated, as in the general structure of an antisense nucleic acid as a nucleic acid drug, and in PT1, the three bases from the 5′ end and the 3′ end have been substituted with LNAs. Moreover, in PT1-3n-1, the three bases from the 5′ end and the two bases from the 3′ end have been substituted with LNAs. Both of PT1 and PT1-3n-1 were prepared and used at 1, 2, 10 or 100 ng/ml using nuclease free water containing 0.01% Tween20. Moreover, a blank sample which did not contain PT1 and PT1-3n-1 was also prepared.

<Base Sequence of PT1>
5′-A(L)\hat{ }G(L)\hat{ }A(L)\hat{ }G\hat{ }C\hat{ }T\hat{ }G\hat{ }A\hat{ }C\hat{ }T\hat{ }T\hat{ }G\hat{ }A\hat{ }T(L)\hat{ }
G(L)\hat{ }5(L)-3′ (The base part is SEQ ID NO: 1.)
<Base Sequence of PT1-3n-1>
5′-A(L)\hat{ }G(L)\hat{ }A(L)\hat{ }G\hat{ }C\hat{ }T\hat{ }G\hat{ }A\hat{ }C\hat{ }T\hat{ }T\hat{ }G\hat{ }A\hat{ }T(L)\hat{ }
G(L)-3′ (The base part is SEQ ID NO: 2.)
* (L) indicates LNA, 5 indicates substitution
with 5-Methyl-Cytosine, and \hat{ } indicates being
phosphorothioated.

### (2) Preparation of Capture Probes

### (2-1) Case Using ECL Plate [Plate-ECL Series] as Solid Phase

By binding a nucleic acid probe having a base sequence complementary to the 3′ side of PT1 [called CP-sp0-B] or one containing a 15-mer oligonucleotide spacer [called CP-sp15-B] onto a plate of MSD GOLD 96-well Small Spot SA SECTOR (MSD/product number: L45SA-1) as a solid phase through biotin modification at the 5′ end, capture probes were prepared [called “plate-ECL-sp0” and “plate-ECL-sp15”, respectively]. The reaction was conducted with Thermomixer comfort (Eppendorf), and after adding a 10 pmol nucleic acid probe to 100 μL of 1×PBS-TP [1×PBS [137 mM Sodium Chloride, 8.1 mM Disodium Phosphate, 2.68 mM Potassium Chloride and 1.47 mM Potassium Dihydrogenphosphate], 0.02% Tween20 and 1.5 ppm ProClin300], the reaction was conducted at 37° C. for 30 minutes with stirring (700 rpm). After washing twice with 200 μL of 1×PBS-TP, 150 μL of 3% BlockerA (MSD, product number: R93BA-4) was added, and the reaction was conducted at 37° C. for an hour (700 rpm). Then, washing was conducted three times with 200 μL of 1×PBS-TP.

<Base Sequence of CP-sp0-B
5′-(biotin)GCATCAAGT-3′ (The base part is
SEQ ID NO: 3.)
<Base Sequence of CP-sp15-B>
5′-(biotin)AGAGTGGTTGTGGTAGCATCAAGT-3′
(The base part is SEQ ID NO: 4.)
* The underlined part indicates the first
spacer site.

### (2-2) Case Using ELOSA Plate [Plate-Elo1 Series] as Solid Phase

By binding the nucleic acid probe CP-sp0-B having a base sequence complementary to the 3′ side of PT1 or CP-sp15-B containing a 15-mer oligonucleotide spacer onto a plate of Pierce (registered trademark) NeutrAvidin (registered trademark) Coated 96-Well Plates (Thermo Fisher Scientific/product number: 15129) as a solid phase through biotin modification at the 5′ end, capture probes were prepared [called “plate-Elo1-sp0” and “plate-Elo1-sp15”, respectively]. The reaction was conducted with Thermomixer comfort (Eppendorf), and after adding a 10 pmol nucleic acid probe to 100 μL of 1×PBS-TP [1×PBS [137 mM Sodium Chloride, 8.1 mM Disodium Phosphate, 2.68 mM Potassium Chloride and 1.47 mM Potassium Dihydrogenphosphate], 0.02% Tween20 and 1.5 ppm ProClin300], the reaction was conducted at 37° C. for 30 minutes (700 rpm). After washing twice with 200 μL of 1×PBS-TP, 150 μL of 3% BlockerA (MSD/product number: R93BA-4) was added, and the reaction was conducted at 37° C. for an hour (700 rpm). Then, washing was conducted three times with 200 μL of 1×PBS-TP.

### (2-3) Case Using ELOSA Plate [Plate-Elo2 Series] as Solid Phase

By binding the nucleic acid probe CP-sp0-B having a base sequence complementary to the 3′ side of PT1 or CP-sp15-B containing a 15-mer oligonucleotide spacer onto a plate of Nunc immobilizer Streptavidin F96 CLEAR (Thermo Fisher Scientific/product number: 436020), which was a solid phase of the type to which an adapter was bonded through a second spacer, through biotin modification at the 5′ end, capture probes were prepared [called “plate-Elo2-sp0” and “plate-Elo2-sp15”, respectively]. The reaction was conducted with Thermomixer comfort (Eppendorf), and after adding a 10 pmol nucleic acid probe to 100 μL of 1×PBS-TP [1×PBS [137 mM Sodium Chloride, 8.1 mM Disodium Phosphate, 2.68 mM Potassium Chloride and 1.47 mM Potassium Dihydrogenphosphate], 0.02% Tween20 and 1.5 ppm ProClin300], the reaction was conducted at 37° C. for 30 minutes (700 rpm). After washing twice with 200 μL of 1×PBS-TP, 150 μL of 3% BlockerA (MSD/product number: R93BA-4) was added, and the reaction was conducted at 37° C. for an hour (700 rpm). Then, washing was conducted three times with 200 μL of 1×PBS-TP.

### (2-4) Case Using Polystyrene Beads [Beads-1 Series] as Solid Phase

By binding the nucleic acid probe CP-sp0-B having a base sequence complementary to the 3′ side of PT1 or CP-sp15-B containing a 15-mer oligonucleotide spacer onto beads of MicroPlex Microspheres (Luminex/product number: LC10015-01) as a solid phase through biotin modification at the 5′ end, capture probes were prepared [called “beads-1-sp0” and “beads-1-sp15”, respectively].

### (2-5) Case Using Polystyrene Beads [Beads-2 Series] as Solid Phase

By binding a nucleic acid probe having a base sequence complementary to the 3′ side of PT1 [called CP-sp0-N] or a nucleic acid probe containing a 15-mer oligonucleotide spacer [called CP-sp15-N] onto beads of MicroPlex Microspheres (Luminex/product number: LC10015-01) as a solid phase through NH₂ modification at the 5′ end, capture probes were prepared [called “beads-2-sp0” and “beads-2-sp15”, respectively].

<Base Sequence of CP-sp0-N>
5′-(NH2)GCATCAAGT-3′ (The base part is SEQ
ID NO: 3.)
<Base Sequence of CP-sp15-N>
5′-(NH₂)AGAGTGGTTGTGGTAGCATCAAGT-3′ (The
base part is SEQ ID NO: 4.)
* The underlined part indicates the first
spacer site.

(3) Capture of Target Nucleic Acid with Capture Probes (1^{st} Hybridization Reaction)

### (3-1) Case Using ECL Plate [Plate-ECL Series] as Solid Phase

To 10 μL of the target nucleic acid, the metabolite model nucleic acid of the target nucleic acid or the blank sample, 40 μL of a 1st hybridization reaction solution was added to provide a total volume of 50 μL, and the reaction was conducted with Thermomixer comfort (Eppendorf) at 25° C. for an hour (700 rpm). The reaction was conducted at 25° C. for an hour.

### (3-1-1) Composition of 1^{st} Hybridization Reaction Solution

5 M TMAC (tetramethylammonium chloride) in a volume of 15 μL, 7.5 μL of 10× supplement [500 mM Tris-HCl (pH 8.0), 40 mM EDTA (pH 8.0) and 8.0% sodium N-lauroylsarcosine], 10 μL of 12.5% PEG8000 (polyethylene glycol), 6.5 μL of RNase free water and 1 μL of 100 fmol/mL assist probe (AP-1 to which a poly A chain was added and which had a base sequence complementary to the 5′ side of PT1).

<Base Sequence of AP-1>
5′-5(L)A(L)G(L)5(L)T(L)5(L)AAAAAAAAAAAAAAAAAAAAAAA
AAAAAAAAAAAAAAAAAAAAAAAAAAAAAAAAAAAAA-3′ (The base
part is SEQ ID NO: 5.)

### (3-2) Case Using ELOSA Plates [Plate-Elo1 Series] [Plate-Elo2 Series] as Solid Phase

To 10 μL of the target nucleic acid, the metabolite model nucleic acid of the target nucleic acid or the blank sample, 90 μL of a 1st hybridization reaction solution was added to provide a total volume of 100 μL, and the reaction was conducted with Thermomixer comfort (Eppendorf) at 25 for an hour (700 rpm).

### (3-2-1) Composition of 1^{st} Hybridization Reaction Solution

5 M TMAC (tetramethylammonium chloride) in a volume of 30 μL, 15 μL of 10× supplement [500 mM Tris-HCl (pH 8.0), 40 mM EDTA (pH 8.0) and 8.0% sodium N-lauroylsarcosine], 20 μL of 12.5% PEG8000 (polyethylene glycol), 24 μL of RNase free water and 1 μL of 100 fmol/mL assist probe (AP-1 (SEQ ID NO:5) to which a poly A chain was added and which had a base sequence complementary to the 5′ side of PT1).

### (3-3) Case Using Polystyrene Beads [Beads-1 Series] [Beads-2 Series] as Solid Phase

To 10 μL of the target nucleic acid, the metabolite model nucleic acid of the target nucleic acid or the blank sample, 25 μL of a 1st hybridization reaction solution was added to provide a total volume of 35 μL, and the reaction was conducted with a thermal cycler (Eppendorf) at 25° C. for an hour.

### (3-3-1) Composition of 1st Hybridization Reaction Solution

A capture probe immobilized in (2) above in a volume of 0.4 μL (800 particles), 10.5 μL of 5 M TMAC (tetramethylammonium chloride), 5.25 μL of 10× supplement [500 mM Tris-HCl (pH 8.0), 40 mM EDTA (pH 8.0) and 8.0% sodium N-lauroylsarcosine], 5 μL of 17.5% PEG8000 (polyethylene glycol), 2.85 μL of RNase free water and 1 μL of 100 fmol/mL assist probe (AP-1 (SEQ ID NO:5) to which a poly A chain was added and which had a base sequence complementary to the 5′ side of PT1).

### (4) Signal Amplification by PALSAR Reaction

### (4-1) Case Using ECL Plate [Plate-ECL Series] as Solid Phase

To 50 μL of the reaction solutions after the 1st hybridization reaction, 30 μL of a PALSAR reaction solution was added to provide a total volume of 80 μL, and the reaction was conducted with Thermomixer comfort (Eppendorf) at 25° C. for an hour. The sequences of the pair of self-assembly probes (also called signal amplification probes) used in the PALSAR reaction are HCP-1-C3 and HCP-2-C3 below, in which the 5′ ends were labeled with cyanine3 (C3).

<Base Sequence of HCP-1-C3>
5′-(C3)CAACAATCAGGACGATACCGATGAAGTTTTTTTTTTTTTTTTT
TTT-3′ (The base part is SEQ ID NO: 6.)
<Base Sequence of HCP-2-C3>
5′-(C3)GTCCTGATTGTTGCTTCATCGGTATCAAAAAAAAAAAAAAAAA
AAA-3′ (The base part is SEQ ID NO: 7.)

### (4-1-1) Composition of PALSAR Reaction Solution

Nuclease-free water in a volume of 5.1 μL, 4.5 μL of 5 M TMAC, 2.25 μL of 10× supplement [500 mM Tris-HCl (pH 8.0), 40 mM EDTA (pH 8.0) and 8% sodium N-lauroylsarcosine], 1.75 μL of 20 pmol/μL HCP-1-C3 and 1.4 μL of 20 pmol/μL HCP-2-C3.

### (4-2) Case Using ELOSA Plates [Plate-Elo1 Series] [Plate-Elo2 Series] as Solid Phase

To 100 μL of the reaction solutions after the 1st hybridization reaction, 50 μL of a PALSAR reaction solution was added to provide a total volume of 150 μL, and the reaction was conducted with Thermomixer comfort (Eppendorf) at 25° C. for an hour (700 rpm). The sequences of the pair of self-assembly probes (also called signal amplification probes) used in the PALSAR reaction are HCP-1-D and HCP-2-D below, in which the 5′ ends were labeled with digoxigenin (Dig).

<Base Sequence of HCP-1-D>
5′-(Dig)CAACAATCAGGACGATACCGATGAAGTTTTTTTTTTTTTTTT
TTTT-3′ (The base part is SEQ ID NO: 6.)
<Base Sequence of HCP-2-D>
5′-(Dig)GTCCTGATTGTTGCTTCATCGGTATCAAAAAAAAAAAAAAAA
AAAA-3′ (The base part is SEQ ID NO: 7.)

### (4-2-1) Composition of PALSAR Reaction Solution

Nuclease-free water in a volume of 24.35 μL, 15 μL of 5 M TMAC, 7.5 μL of 10× supplement [500 mM Tris-HCl (pH 8.0), 40 mM EDTA (pH 8.0) and 8% sodium N-lauroylsarcosine], 1.75 μL of 20 pmol/μL HCP-1-D and 1.4 μL of 20 pmol/μL HCP-2-D.

### (4-3) Case Using Polystyrene Beads [Beads-1 Series] as Solid Phase

To 35 μL of the reaction solutions after the 1st hybridization reaction, 15 μL of a PALSAR reaction solution was added to provide a total volume of 50 μL, and the reaction was conducted with a Thermal cycler (Eppendorf) at 25° C. for an hour. The sequences of the pair of self-assembly probes (also called signal amplification probes) used in the PALSAR reaction are HCP-1-C3 (SEQ ID NO: 6) and HCP-2-C3 (SEQ ID NO: 7), in which the 5′ ends were labeled with cyanine3 (C3).

### (4-3-1) Composition of PALSAR Reaction Solution

Nuclease-free water in a volume of 5.1 μL, 4.5 μL of 5 M TMAC, 2.25 μL of 10× supplement [500 mM Tris-HCl (pH 8.0), 40 mM EDTA (pH 8.0) and 8% sodium N-lauroylsarcosine], 1.75 μL of 20 pmol/μL HCP-1-C3 and 1.4 μL of 20 pmol/μL HCP-2-C3.

### (4-4) Case Using Polystyrene Beads [Beads-2 Series] as Solid Phase

To 35 μL of the reaction solutions after the 1st hybridization reaction, 15 μL of a PALSAR reaction solution was added to provide a total volume of 50 μL, and the reaction was conducted with a Thermal cycler (Eppendorf) at 25° C. for an hour. The sequences of the pair of self-assembly probes (also called signal amplification probes) used in the PALSAR reaction are HCP-1-D (SEQ ID NO:6) and HCP-2-D (SEQ ID NO:7), in which the 5′ ends were labeled with digoxigenin (Dig).

### (4-3-1) Composition of PALSAR Reaction Solution

Nuclease-free water in a volume of 4.6 μL, 4.5 μL of 5 M TMAC, 2.75 μL of 10× supplement [500 mM Tris-HCl (pH 8.0), 40 mM EDTA (pH 8.0) and 8% sodium N-lauroylsarcosine], 1.75 μL of 20 pmol/μL HCP-1-D and 1.4 μL of 20 pmol/μL HCP-2-D.

### (5) Detection

### (5-1) Case Using ECL Plate [Plate-ECL Series] as Solid Phase

Washing was conducted twice with 200 μL of 1×PBS-TP after the completion of the PALSAR reaction. Then, 0.25 μL of an anti-Dig-Fab antibody-Ru (MSD SULFO-TAG NHS-Ester; MSD/product number: R91AO-1, Anti-DIG, Fab fragments from sheep; Roche/product number: 11214667001) which was self-prepared at 100 μg/mL, 16.7 μL of 3% BlockerA and 33.1 μL of nuclease-free water were added, and the reaction was conducted with Thermomixer comfort at 25° C. for an hour (700 rpm). After washing again three times with 200 μL of 1×PBS-TP, 150 μL of MSD GOLD Read Buffer A (MSD/product number: R92TG-2) which was diluted 2-fold with nuclease-free water was added, and the positive signal values (PS) and the background values (BG) of the target nucleic acid and the metabolite model nucleic acid were measured with a MESO QuickPlex SQ 120 MM measurement device (MSD).

### (5-2) Case Using ELOSA Plates [Plate-Elo1 Series] [Plate-Elo2 Series] as Solid Phase

Washing was conducted three times with 200 μL of 1×PBS-TP after the completion of the PALSAR reaction. Then, 100 μL of an anti-Dig-HRP antibody (R&Dsystems/product number: HAM7520) which was diluted 10000-fold with 1×PBS-TP containing 1% BSA was added, and the reaction was conducted with Thermomixer comfort at 25° C. for an hour (700 rpm). After washing three times with 200 μL of 1×PBS-TP, 100 μL of a luminescent substrate solution (KPL SureBlue Reserve TMB Microwell Peroxidase Substrate; seracare/product number: 5120-0083) was added, and after washing again three times with 200 μL of 1×PBS-TP, 100 μL of a reaction stop solution (1 M sulfuric acid/product number 198-09595) was added. The positive signal values (PS) and the background values (BG) of the target nucleic acid and the metabolite model nucleic acid were measured using TECAN infinite50R; 50-620 nm wavelength (TECAN).

### (5-3) Case Using Polystyrene Beads [Beads-1 Series] as Solid Phase

The reaction solutions after the completion of the PALSAR reaction were washed twice with 1×PBS-TP [1×PBS [137 mM Sodium Chloride, 8.1 mM Disodium Phosphate, 2.68 mM Potassium Chloride and 1.47 mM Potassium Dihydrogenphosphate], 0.02% Tween20 and 1.5 ppm ProClin300].

Then, 75 μL of 1×PBS-TP was added, and the positive signal values (PS) and the background values (BG) of the target nucleic acid and the metabolite model nucleic acid were measured using Luminex System (Luminex).

### (5-4) Case Using Polystyrene Beads [Beads-2 Series] as Solid Phase

After the completion of the PALSAR reaction, washing was conducted once with 1×PBS-TP [1×PBS [137 mM Sodium Chloride, 8.1 mM Disodium Phosphate, 2.68 mM Potassium Chloride and 1.47 mM Potassium Dihydrogenphosphate], 0.02% Tween20 and 1.5 ppm ProClin300]. A 5 μg/mL detection reagent (SAPE; Streptavidin-R-Phycoerythrin, Prozyme/product number: PJ31S-1) in a volume of 50 μL was added, and the mixtures were left still with shading at 25° C. for 10 minutes and then washed twice with 1×PBS-TP. Then, 75 μL of 1×PBS-TP was added, and the positive signal values (PS) and the background values (BG) of the target nucleic acid and the metabolite model nucleic acid were measured using Luminex System (Luminex).

### 2. Results

### (1) Examination Using ECL Plate [Plate-ECL Series] as Solid Phase

The target nucleic acid and the metabolite model were measured using the capture probe “plate-ECL-sp0” having no first spacer and the capture probe “plate-ECL-sp15” having a first spacer, and the cross-reactivities are shown in Table 1. The average cross-reactivity was 44.0% when “plate-ECL-sp0” having no first spacer was used as the capture probe, while the average cross-reactivity was less than 1% when “plate-ECL-sp15”, into which a first spacer was introduced, was used. Thus, it was shown that the cross-reactivity can be suppressed significantly by inserting a first spacer into the capture probe.

TABLE 1

| | |
|---|---|
| Target Nucleic | Cross-Reactivity (%) |

| Acid (ng/mL) | plate-ECL-sp0 | plate-ECL-sp15 |
|---|---|---|
| 1 | 49.4 | −1.1 |
| 10 | 38.5 | 1.4 |
| Average Cross- | 44.0 | <1% |
| Reactivity | | |

Cross-Reactivity (%) = [Metabolite Model Nucleic Acid (PS-BG)]/[Target Nucleic Acid (PS-BG)] × 100

### (2) Examination Using ELOSA Plate [Plate-Elo1 Series] as Solid Phase

The target nucleic acid and the metabolite model were measured using the capture probe “plate-Elo1-sp0” having no first spacer and the capture probe “plate-Elo1-sp15” having a first spacer, and the cross-reactivities are shown in Table 2. The average cross-reactivity was 111.8% when “plate-Elo1-sp0” having no first spacer was used as the capture probe, while the average cross-reactivity was 3.9% when “plate-Elo1-sp15”, into which a first spacer was introduced, was used. Thus, it was shown that the cross-reactivity can be suppressed significantly by inserting a first spacer into the capture probe.

TABLE 2

| | |
|---|---|
| Target Nucleic | Cross-Reactivity (%) |

| Acid (ng/mL) | plate-Elo1-sp0 | plate-Elo1-sp15 |
|---|---|---|
| 10 | 117.4 | 0.4 |
| 100 | 106.2 | 7.4 |
| Average Cross- | 111.8 | 3.9 |
| Reactivity | | |

Cross-Reactivity (%) = [Metabolite Model (PS-BG)]/[Target Nucleic Acid (PS-BG)] × 100

### (3) Examination Using ELOSA Plate [Plate-Elo2 Series] as Solid Phase

The target nucleic acid and the metabolite model were measured using the capture probe “plate-Elo2-sp0” having no first spacer and the capture probe “plate-Elo2-sp15” having a first spacer, and the cross-reactivities are shown in Table 3. The average cross-reactivity was 55.5% when “plate-Elo2-sp0” having no first spacer was used as the capture probe, while the average cross-reactivity was less than 1% when “plate-Elo2-sp15”, into which a first spacer was introduced, was used. It was thus shown that the cross-reactivity can be suppressed significantly by inserting a first spacer into the capture probe.

TABLE 3

| | |
|---|---|
| Target Nucleic | Cross-Reactivity (%) |

| Acid (ng/mL) | plate-Elo2-sp0 | plate-Elo2-sp15 |
|---|---|---|
| 1 | 13.8 | −0.4 |
| 10 | 97.2 | 1.3 |
| Average Cross- | 55.5 | <1% |
| Reactivity | | |

Cross-Reactivity (%) = [Metabolite Model (PS-BG)]/[Target Nucleic Acid (PS-BG)] × 100

### (4) Examination Using Polystyrene Beads [Beads-1 Series] as Solid Phase

The target nucleic acid and the metabolite model were measured using the capture probe “beads-1-sp0” having no first spacer and the capture probe “beads-1-sp15” having a first spacer, and the cross-reactivities are shown in Table 4. The average cross-reactivity was 29.1% when “beads-1-sp0” having no first spacer was used as the capture probe, while the average cross-reactivity was 3.7% when “beads-1-sp15”, into which a first spacer was introduced, was used. Thus, it was shown that the cross-reactivity can be suppressed significantly by inserting a first spacer into the

TABLE 4

| | |
|---|---|
| Target Nucleic | Cross-Reactivity (%) |

| Acid (ng/mL) | beads-1-sp0 | beads-1-sp15 |
|---|---|---|
| 1 | 6.4 | 1.5 |
| 10 | 51.7 | 6 |
| Average Cross- | 29.1 | 3.7 |
| Reactivity | | |

Cross-Reactivity (%) = [Metabolite Model (PS-BG)]/[Target Nucleic Acid (PS-BG)] × 100

### (5) Examination Using Polystyrene Beads [Beads-2 Series] as Solid Phase

The target nucleic acid and the metabolite model were measured using the capture probe “beads-2-sp0” having no first spacer and the capture probe “beads-2-sp15” having a first spacer, and the cross-reactivities are shown in Table 5. The average cross-reactivity was 1.6% when “beads-2-sp0” having no first spacer was used as the capture probe, while the average cross-reactivity was less than 1% when “beads-2-sp15”, into which a first spacer was introduced, was used. Thus, it was shown that the cross-reactivity can be suppressed by inserting a first spacer into the

TABLE 5

| | |
|---|---|
| Target Nucleic | Cross-Reactivity (%) |

| Acid (ng/mL) | beads-2-sp0 | beads-2-sp15 |
|---|---|---|
| 1 | 0.7 | −0.1 |
| 2 | 2.7 | 1.6 |
| Average Cross- | 1.6 | <1% |
| Reactivity | | |

Cross-Reactivity (%) = [Metabolite Model (PS-BG)]/[Target Nucleic Acid (PS-BG)] × 100

### [Example 2] Examination of Length of Spacer Between Solid Phase and Nucleic Acid Probe Contained in Capture Probe

### 1. Materials and Methods

### (1) Target Nucleic Acid

The target nucleic acid PT1 (SEQ ID NO: 1) and the metabolite model nucleic acid (a nucleic acid which lacked one base at the 3′ end) PT1-3n-1 (SEQ ID NO: 2) of the target nucleic acid used in Example 1 were used. Both of PT1 and PT1-3n-1 were prepared and used at 10 ng/mL using nuclease free water containing 0.01% Tween20. Moreover, a blank sample which did not contain PT1 and PT1-3n-1 was also prepared.

### (2) Preparation of Capture Probes

By binding a nucleic acid probe CP-sp0-B having a base sequence complementary to the 3′ side of PT1 and containing no spacer or CP-sp5-B, CP-sp10-B, CP-sp15-B, CP-sp20-B, CP-sp25-B, CP-sp30-B, CP-sp50-B or CP-sp90-B containing a 5-, 10-, 15-, 20-, 25-, 30-, 50- or 90-mer oligonucleotide spacer onto a plate of MSD GOLD 96-well Small Spot SA SECTOR (MSD/product number: L45SA-1) as a solid phase through biotin modification at the 5′ end, capture probes were prepared. The reaction was conducted with Thermomixer comfort (Eppendorf), and after adding a 10 pmol nucleic acid probe to 100 μL of 1×PBS-TP [1×PBS [137 mM Sodium Chloride, 8.1 mM Disodium Phosphate, 2.68 mM Potassium Chloride and 1.47 mM Potassium Dihydrogenphosphate], 0.02% Tween20 and 1.5 ppm ProClin300], the reaction was conducted at 37° C. for 30 minutes (700 rpm). After washing twice with 200 μL of 1×PBS-TP, 150 μL of 3% BlockerA (MSD, product number: R93BA-4) was added, and the reaction was conducted at 37° C. for an hour (700 rpm). Then, washing was conducted three times with 200 μL of 1×PBS-TP.

<Base Sequence of CP-sp0-B>
5′-(biotin)GCATCAAGT-3′ (The base part is SEQ ID
NO: 3.)
<Base Sequence of CP-sp5-B>
5′-(biotin)TGGTAGCATCAAGT-3′ (The base part is SEQ
ID NO: 8.)
<Base Sequence of CP-sp10-B>
5′-(biotin)GGTTGTGGTAGCATCAAGT-3′ (The base part
is SEQ ID NO: 9.)
<Base Sequence of CP-sp15-B>
5′-(biotin)AGAGTGGTTGTGGTAGCATCAAGT-3′ (The base
part is SEQ ID NO: 4.)
<Base Sequence of CP-sp20-B
5′-(biotin)TGGTAAGAGTGGTTGTGGTAGCATCAAGT-3′ (The
base part is SEQ ID NO: 10.)
<Base Sequence of CP-sp25-B>
5′-(biotin)AGAGTTGGTAAGAGTGGTTGTGGTAGCATCAAGT-3′
(The base part is SEQ ID NO: 11.)
<Base Sequence of CP-sp30-B>
5′-(biotin)TTGTGAGAGTTGGTAAGAGTGGTTGTGGTAGCATCAAG
T-3′ (The base part is SEQ ID NO: 12.)
<Base Sequence of CP-sp50-B>
5′-(biotin)TGGTAAGAGTGGTTGTGGTATTGTGAGAGTTGGTAAGAG
TGGTTGTGGTAGCATCAAGT-3′ (The base part is SEQ ID
NO: 13.)
<Base Sequence of CP-sp90-B>
5′-(biotin)GGTTGTGGTATTGTGAGAGTTGGTAAGAGTGGTTGTGGT
ATGGTAAGAGTGGTTGTGGTATTGTGAGAGTTGGTAAGAGTGGTTGTGGT
AGCATCAAGT-3′ (The base part is SEQ ID NO: 14.)
* The underlined part indicates the first spacer
site.

(3) Capture of Target Nucleic Acid with Capture Probes (1^{st} Hybridization Reaction)

To 10 μL of the target nucleic acid, the metabolite model nucleic acid of the target nucleic acid or the blank sample, 40 μL of a 1st hybridization reaction solution was added to provide a total volume of 50 μL, and the reaction was conducted with Thermomixer comfort (Eppendorf) at 25° C. for an hour (700 rpm). The reaction was conducted at 25° C. for an hour.

### (3-1) Composition of 1^{st} Hybridization Reaction Solution

5 M TMAC (tetramethylammonium chloride) in a volume of 15 μL, 7.5 μL of 10× supplement [500 mM Tris-HCl (pH 8.0), 40 mM EDTA (pH 8.0) and 8.0% sodium N-lauroylsarcosine], 10 μL of 12.5% PEG8000 (polyethylene glycol), 6.5 μL of RNase free water and 1 μL of 100 fmol/mL assist probe (AP-1 (SEQ ID NO:5) to which a poly A chain was added and which had a base sequence complementary to the 5′ side of PT1).

### (4) Signal Amplification by PALSAR Reaction

To 50 μL of the reaction solutions after the 1st hybridization reaction, 30 μL of a PALSAR reaction solution was added to provide a total volume of 80 μL, and the reaction was conducted with Thermomixer comfort (Eppendorf) at 25° C. for an hour. The sequences of the pair of self-assembly probes (also called signal amplification probes) used in the PALSAR reaction are HCP-1-C3 (SEQ ID NO: 6) and HCP-2-C3 (SEQ ID NO: 7), in which the 5′ ends were labeled with cyanine3 (C3).

### (4-1) Composition of PALSAR Reaction Solution

Nuclease-free water in a volume of 5.1 μL, 4.5 μL of 5 M TMAC, 2.25 μL of 10× supplement [500 mM Tris-HCl (pH 8.0), 40 mM EDTA (pH 8.0) and 8% sodium N-lauroylsarcosine], 1.75 μL of 20 pmol/μL HCP-1-C3 and 1.4 μL of 20 pmol/μL HCP-2-C3.

### (5) Detection

Washing was conducted twice with 200 μL of 1×PBS-TP after the completion of the PALSAR reaction. Then, 0.25 μL of an anti-Dig-Fab antibody-Ru (MSD SULFO-TAG NHS-Ester; MSD/product number: R91AO-1, Anti-DIG, Fab fragments from sheep; Roche/product number: 11214667001) which was self-prepared at 100 μg/mL, 16.7 μL of 3% BlockerA and 33.1 μL of nuclease-free water were added, and the reaction was conducted with Thermomixer comfort at 25° C. for an hour (700 rpm). After washing again three times with 200 μL of 1×PBS-TP, 150 μL of MSD GOLD Read Buffer A (MSD/product number: R92TG-2) which was diluted 2-fold with nuclease-free water was added, and the positive signal values (PS) and the background values (BG) of the target nucleic acid and the metabolite model nucleic acid were measured with a MESO QuickPlex SQ 120 MM measurement device (MSD).

### 2. Results

The target nucleic acid and the metabolite model were measured using the capture probe (with CP-sp0-B) having no first spacer and the capture probes (with CP-sp5-B, CP-sp10-B, CP-sp15-B, CP-sp20-B, CP-sp25-B, CP-sp30-B, CP-sp50-B and CP-sp90-B) having 5-, 10-, 15-, 20-, 25-, 30-, 50- and 90-mer first spacers, respectively, and the cross-reactivities are shown in Table 6. The cross-reactivity was 24.8% when the capture probe did not have a first spacer: while the cross-reactivity was 5.9% when a 5-mer spacer was inserted into the CP; the cross-reactivity was less than 1% when a 10-mer, 15-mer, 20-mer, 25-mer or 30-mer spacer was inserted; the cross-reactivity was 1.4% when a 50-mer spacer was inserted; and the cross-reactivity was 4.8% when a 90-mer spacer was inserted. By extrapolation from the above results, it was shown that the cross-reactivity becomes 10% or less and can be suppressed significantly by inserting a first spacer of around 4-mer to 130-mer into the capture probe.

TABLE 6

| Spacer Length (mer) | Cross-Reactivity |
|---|---|
| 0 (none) | 24.8% |
| 5 | 5.9% |
| 10 | <1% |
| 15 | <1% |
| 20 | <1% |
| 25 | <1% |
| 30 | <1% |
| 50 | 1.4% |
| 90 | 4.8% |

Cross-Reactivity (%) = [Metabolite Model Nucleic Acid (PS-BG)]/[Target Nucleic Acid (PS-BG)] × 100

### INDUSTRIAL APPLICABILITY

Using the measurement method of the invention, the drug concentration in a biological sample of an animal or a human to which the drug has been administered can be measured accurately without being influenced by a metabolite, in a pharmacokinetics/pharmacodynamics (PK/PD) screening test in the searching stage of drug development, in a safety test, a pharmacological test and a pharmacokinetics test in the non-clinical stage and in the clinical stage.

1. (canceled)

2. A method for detecting a target oligonucleotide in a sample while distinguishing from a metabolite thereof which lacks one or more nucleotides from the 3′ end or the 5′ end, comprising:
(i) bringing a capture probe for capturing the target oligonucleotide and an assist probe for detecting the target oligonucleotide into contact with a sample containing the target oligonucleotide or the metabolite thereof which lacks one or more nucleotides from the 3′ end or the 5′ end and forming a complex of the capture probe, the target oligonucleotide and the assist probe,
wherein
the capture probe contains a solid phase, a first nucleic acid probe immobilized on the solid phase and a first spacer between the solid phase and the first nucleic acid probe,
the assist probe contains a tag or a label and a second nucleic acid probe linked to the tag or the label,
the sequence of the first nucleic acid probe is complementary to a partial sequence of the target oligonucleotide, wherein the partial sequence contains the one or more nucleotides which are lacking in the metabolite, and
the sequence of the second nucleic acid probe is complementary to a sequence in the target oligonucleotide other than the partial sequence; and
(ii) detecting the target oligonucleotide in the sample by detecting the complex.

3. A method for detecting a target oligonucleotide in a sample, comprising:
(i) bringing a capture probe for capturing the target oligonucleotide and an assist probe for detecting the target oligonucleotide into contact with a sample and forming a complex of the capture probe, the target oligonucleotide and the assist probe,
wherein
the capture probe contains a solid phase, a first nucleic acid probe immobilized on the solid phase and a first spacer between the solid phase and the first nucleic acid probe,
the assist probe contains a tag or a label and a second nucleic acid probe linked to the tag or the label,
the sequence of the first nucleic acid probe is complementary to a partial sequence of the target oligonucleotide including the nucleotide at an end of the target oligonucleotide, and
the sequence of the second nucleic acid probe is complementary to a sequence in the target oligonucleotide other than the partial sequence; and
(ii) detecting the target oligonucleotide in the sample by detecting the complex.

4. The method according to claim 2, wherein the capture probe contains an adapter between the solid phase and the first spacer.

5. The method according to claim 4, wherein the capture probe contains a second spacer between the solid phase and the adapter.

6. The method according to claim 4, wherein the adapter is streptavidin or avidin and biotin.

7. The method according to claim 6, wherein the binding of the streptavidin or the avidin and the biotin occurs after the formation of the complex of the capture probe, the target oligonucleotide and the assist probe.

8. The method according to claim 2, wherein the first spacer is a 5-mer to 90-mer oligonucleotide.

9. A detection kit for use in detecting a target oligonucleotide, comprising a capture probe, an assist probe and a pair of signal amplification probes which has complementary base sequence regions that can hybridize to each other and which is capable of forming a probe polymer through self-assembly,
wherein
the capture probe contains a solid phase, a first nucleic acid probe immobilized on the solid phase and a first spacer between the solid phase and the first nucleic acid probe,
the assist probe contains a tag having a base sequence which is complementary to a part of or the whole of one signal amplification probe of the pair of signal amplification probes and a second nucleic acid probe linked to the tag,
the sequence of the first nucleic acid probe is complementary to a partial sequence of the target oligonucleotide including the nucleotide at an end of the target oligonucleotide, and
the sequence of the second nucleic acid probe is complementary to a sequence in the target oligonucleotide other than the partial sequence.

10. The detection kit according to claim 9, wherein the capture probe contains an adapter between the solid phase and the first spacer.

11. The detection kit according to claim 10, wherein the capture probe contains a second spacer between the solid phase and the adapter.

12. The detection kit according to claim 10, wherein the adapter is streptavidin or avidin and biotin.

13. The detection kit according to claim 12, wherein the binding of the streptavidin or the avidin and the biotin occurs after the formation of the complex of the capture probe, the target oligonucleotide and the assist probe.

14. The detection kit according to claim 9, wherein the first spacer is a 5-mer to 90-mer oligonucleotide.

15. The detection kit according to claim 9, wherein at least one of the pair of signal amplification probes is labeled with a labeling substance.

16. The detection kit according to claim 9,
wherein
the pair of signal amplification probes contains a first signal amplification probe and a second signal amplification probe,
the first signal amplification probe is a nucleic acid probe containing at least a nucleic acid region X, a nucleic acid region Y and a nucleic acid region Z or a nucleic acid region Z containing a poly T sequence in this order from the 5′ end side, and
the second signal amplification probe is a nucleic acid probe containing at least a nucleic acid region X′ which is complementary to the nucleic acid region X, a nucleic acid region Y′ which is complementary to the nucleic acid region Y and a nucleic acid region Z′ which is complementary to the nucleic acid region Z or a nucleic acid region Z′ containing a poly A sequence in this order from the 5′ end side.

17. The method according to claim 3, wherein the capture probe contains an adapter between the solid phase and the first spacer.

18. The method according to claim 5, wherein the adapter is streptavidin or avidin and biotin.

19. The method according to claim 3, wherein the first spacer is a 5-mer to 90-mer oligonucleotide.

20. The method according to claim 4, wherein the first spacer is a 5-mer to 90-mer oligonucleotide.

21. The method according to claim 5, wherein the first spacer is a 5-mer to 90-mer oligonucleotide.
